(19) United States
(12) Patent Application Publication
(10) Pub. No.: US 2024/0358597 A1
(43) Pub. Date: Oct. 31, 2024

(54) LIVE MONITORING PILL DISPENSING DEVICE

(71) Applicant: MEDIFORM TECHNOLOGY CORP., Dover, DE (US)

(72) Inventors: Aden WHITWORTH, Seattle, WA (US); Jack CAMPBELL, West Vancouver (CA)

(21) Appl. No.: 18/684,504

(22) PCT Filed: Aug. 4, 2022
(86) PCT No.: PCT/CA2022/051192

§ 371 Date: Feb. 16, 2024

Related U.S. Application Data
(60) Provisional application No. 63/233,379, filed on Aug. 16, 2021.

(51) Int. Cl. A61J 7/02; A61J 1/14; A61J 1/18; A61J 7/04

(52) U.S. Cl. CPC A61J 7/02; A61J 1/1437; A61J 1/18; A61J 7/0409; A61J 2200/70

(57) ABSTRACT

A tamper-resistant pill dispensing device that utilizes live data to regulate pill consumption and send virtual flags to physicians if patterns of dependence are triggered. The device contains two separate components. The first is a live monitoring dispensing device assembly that detects and collects data as to whether the device has been tampered with. This smart device uses a microcontroller and timer to track data in real time and control circuitry to release pills during the exact times the physician instructed. The second component is a disposable capsule assembly that is compatible with current pharmacist pill-loading practices. The disposable capsule assembly attaches to the dispensing device assembly and once connected cannot be disconnected until instructed by a physician. Once connected, a patient can use various user interfaces and physical devices to better regulate their prescription pill consumption.

### FIELD OF THE INVENTION

The disclosed invention relates to medical devices and systems for monitoring patients' adherence to prescribed opioid prescriptions and for limiting access to the prescribed pills. The device may be reused, and is physician and pharmacist friendly. Compliance with the predefined dosage intervals is monitored, which gives physicians the information needed to make informed decisions on future prescriptions.

### BACKGROUND OF THE RELATED ART

It is contemplated that the disclosed invention may find particular application in connection with prescription opioids for pain treatment, and is thus generally discussed and illustrated herein in such context. However, it should be appreciated that the present invention may be used in other scenarios in which medication may be subject to potential abuse or misuse, and where monitoring of medication by patients may make sense or be beneficial.

Prescription opioids are generally used to treat moderate to severe pain. Depending on whether the patient's pain is acute or chronic, a physician may decide to prescribe differently. Acute pain happens fast, and typically refers to a sharp pain that temporarily lasts less than six months. Such pain is caused by a specific bodily compromise such as a broken bone. Acute pain patients are traditionally only prescribed opioids when pain is severe. Chronic pain on the other hand affects patients who have extended pain for longer than six months. Such pain is often caused by an underlying issue that may not be treatable. In 2018, the Centers for Disease Control and Prevention (“CDC”) reported that just over twenty percent of Americans suffer from some form of chronic pain. Typical opioid prescriptions for both acute and/or chronic pain are oxycodone, hydrocodone, morphine, and fentanyl.

The CDC reported based on 2014 statistics that the probability of obtaining a refill for acute pain opioid prescriptions was around twenty five percent. Chronic pain patients are usually given thirty day supplies, meaning that they might fill/refill an opioid prescription up to six times. As prescription refill amounts increase, so do patients' time on the drugs. The CDC stated that roughly 25 percent of patients prescribed prescription opioids for chronic pain misuse them, and roughly 10 percent of patients using an opioid for chronic pain developed an opioid disorder.

Currently, prescription opioids are still being freely given to patients with minimal monitoring of compliance to ensure physician's instructions are being followed appropriately. With such a large percentage of patients potentially abusing or misusing, one would think that opioids would be heavily monitored. This is not the case, and instead prescriptions are handed directly to the patient with no check-ins or supervision. This can cause many indirect problems, such as over-prescribing which can lead to an overdose or increased risk of addiction. Additionally, many individuals who become dependent or addicted to prescription opioids may transition into a more steady supply of opiates such as heroin. Another problem with not monitoring prescription opioids is the risk of the prescription being sold on the black market or ingested by unintended individuals.

In terms of related prior art, U.S. patent Ser. No. 16/161,965 (Publication No. 2019/004612, to Brady et al.) discloses a device that can monitor when a user interacts with the medications but it relies solely on the use of a tablet disk to hold the medication. Brady's tablet disk is not practical for holding large prescriptions of one hundred or more pills due to the size limitation of the device. Patients will not want to carry around a device the size of their portable computer. Another disadvantage of Brady's device is that the tablet disk does not offer a practical means of loading or filling. Every prescription would have to be manufactured specifically for their device and for the tablets. This is vastly different from the current procedure of filling prescriptions at a pharmacy, which will make going to market difficult and expensive.

U.S. Pat. No. 10,709,643 (Publication No. 2020/0170889 to Hsu) discloses another device that restricts and monitors a patient's usage, but it destroys the pills when tampered with. A major disadvantage of Hsu's device is that it has an impractical means of filling the prescription. A magazine-like mechanism is used in Hsu's device, which would require very particular orientation and excess time to load. It is not practical to expect pharmacists to add extra time to their day to just fill Hsu's specific device's magazine. Additionally, Hsu's device has to be opened up in order to gain access to the magazine which will take even more work for the pharmacist to do. Because Hsu's device is impractical to load or fill the prescription, that device offers a solution that would have to be completely replaced for each prescription. This means either completely disposing of the device or disabling it. A further disadvantage of Hsu's device is that it requires patients to use their monitoring device in conjunction with each pill taken. This requires that users take unnecessary steps to receive their pills, and it means that their device cannot function without constant communication to the monitoring device. Additionally, Hsu's monitoring device detects that the user hasn't taken their dosage within a short period of time, then it will deny the user access to the medication. This is not practical because not every user will be able to access the medication at the exact time, and the device would do more harm than help as the patient would have to re-wait for an extended period of time. As such, the prior art has various limitations.

### BRIEF SUMMARY OF THE INVENTION

Disclosed herein is an improved design for a pill dispensing device which may at any moment track a patient's behavior when using their prescription.

The present invention not only solves the problem of regulating the distribution of pills to a patient, but it also gives medical professionals the ability to examine how a patient interacts with their prescriptions. In at least one embodiment, one tamper-resistant disposable capsule assembly can be inserted into one dispensing device assembly, allowing for the prescribed number of pills to be taken after initiation by the user interface. The user interface allows for the details of the patient's database access to be sent to the dispensing device where it is stored in memory. Such prescription details can include database access codes and patient specific unique identifiers for database access to the patient's prescription information. The dispensing device itself communicates with the database and accesses the prescription information. The prescription information can detail various parameters such as the number of pills in the prescription, time interval between each dosage, number of pills allowed at each dosage, if any additional pills have been allowed, etc. Upon the patient device's successful communication to the database, the first dosage of the prescription is made available to the patient. This occurs when the solenoid actuator releases the button and the spring returns the button to the starting position. At this point, the patient may press the button and the dosage will fall out of the device. The button is locked in the bottom position due the same solenoid actuator restricting its movement. When the prescribed time has passed by, the same process of releasing and taking the pill will occur. The pills do not need to be taken as soon as the device allows the button to be pushed, but instead the dispensing device and system monitors both the time the button was released and the time it was pressed. If additional pills have been requested and approved, then the dispensing device would allow for the button to be pressed again immediately after the normally prescribed dosage.

In a preferred embodiment, the device has two power sources which allows for the dispensing device to have unique advantages. One power source is unique to the main operations of the device, while the other power source is only used for the timer. This offers the device the capability to continue to keep time even if the device does not have power.

Other devices offer solutions that require a specific magazine or packaging to be filled in an unnatural way for health professionals. For example, a magazine-like device would require each individual pill to be pressed into such a device by a pharmacist, or pre determined packaging would require each prescription to be manufactured with the specific patient's pills inside before sending it to the patient. The disclosed tamper resistant capsules allow medical professionals to continue the same way of filling a prescription as done today with the orange canisters, and does not change any processes in the supply chain of the prescription filling process. All that is required to fill the disposable capsule assemblies is for all the pills to be poured into the tamper-resistant capsules and place a locking lid on top. The dispensing device's operation allows for repeat use of the dispensing device assembly in accordance with new disposable capsules assemblies for each prescription prescribed to a patient.

In accordance with one aspect of the present invention, disclosed herein is a pill dispensing device for managing the dispensing of prescribed pills to a patient, the pill dispensing device comprising: a pill dispensing assembly; and a tamper-resistant disposable capsule assembly, wherein the disposable capsule assembly and the pill dispensing assembly are configured to engage with each other in an engaged configuration, wherein the pill dispensing assembly comprises: (a) a dispensing button for dispensing the pills; (b) an electronic circuit comprising: (i) a microcontroller with memory; (ii) a first power supply for powering the microcontroller; (iii) a timer; (iv) a solenoid actuator configured to lock or unlock the dispensing button; (v) a sensor configured to detect when the dispensing button has been pressed and when a pill has been dispensed; and (vi) communication means; and (c) a patient user interface configured to receive input from a patient and configured to communicate prescription information to the patient; wherein the microcontroller is configured to store dispensing device data in the memory; wherein the microcontroller is configured to actuate the solenoid actuator according to the dispensing device data; wherein the microcontroller is configured to communicate the dispensing device data via the communication means to an external patient database; and wherein the disposable capsule assembly comprises: a housing for containing a plurality of pills; a locking lid; a trap door configured to open in response to the patient pressing the dispensing button, thereby allowing one or more of the plurality of pills to be dispensed from the dispensing device to the patient.

In some aspects, the dispensing device data includes one or more of prescription data, patient data, time from the timer, name of prescribed drug, amount of pills in a total prescription, prescription dosage (number of pills in a dose), frequency of dispensing each dose, time period between respective doses, prescription frequency, date and time of prescription activation, time/date when each dose is made available to the patient, time/date when a dose is dispensed to the patient, expiry of prescription, and time for renewing a prescription,

In some aspects, the microcontroller is configured to actuate the solenoid actuator and unlock the dispensing button according to the time/date when each dose is to be made available to the patient. In yet other aspects, the dispensing deice is configured to lock the dispensing button, once a pill or a dose of pills have been dispensed.

In some aspects of the present invention, the pills are opioids.

In some aspects, the electronic circuit additionally comprises a second power supply for powering the timer.

In yet other aspects, the power to operate the microcontroller is supplied only when the disposable capsule assembly and dispensing device assembly are properly engaged in the engaged position.

In some aspects, the dispensing device is configured, in response to the patient pressing the dispensing button when it is unlocked, to dispense a single dose of the plurality of pills.

In yet other aspects, the dispensing device is additionally provided with means for sensing whether the integrity of the dispensing capsule assembly has been compromised.

In yet another aspect of the present invention, the patient user interface is configured to permit the patient to submit an electronic request via the communication means to a physician for an additional or exceptional dosage, and the dispensing device is configured to unlock the dispensing button in the event of the physician's approval of such electronic request.

In accordance with another aspect of the present invention, disclosed herein is a pill dispensing system for managing the dispensing of pills to a patient, the system comprising the pill dispensing device, and an external patient database in communication with the dispensing device, wherein the microcontroller of the dispensing device is configured to lock or unlock the dispensing button of the dispensing device according to the dispensing device data.

Also disclosed herein is a corresponding method of managing the dispensing of pills to a patient, as well as a use of the dispensing device.

Additional features, objects, and advantages of the present disclosure will become readily apparent from the following description, drawings, and claims.

### BRIEF DESCRIPTION OF THE DRAWINGS

The accompanying schematic drawings and flow diagrams reference the numerous embodiments of the disclosed invention. Corresponding figures reference various parts of the disclosed invention, where:

FIGS. 1A-1B is a flow diagram illustrating the mechanics of how the pill dispensing device operates with the patient and the database as disclosed herein.

FIGS. 2A-2B is a flow diagram illustrating the mechanics of how the pill dispensing device's user interface interacts with the dispensing device, database, patients, and medical professionals as disclosed herein.

FIG. 3 is a flow diagram describing the communication protocols between the user interface, database, and the dispensing device as disclosed herein.

FIG. 4 is a high level front perspective view of the of the electronic circuit associated with the dispensing device as disclosed herein.

FIGS. 5A-50 are schematic diagrams of an example embodiment of the pill dispensing device's user interface, showing a request for additional pill, prescription analytics, and unlocking protocols.

FIGS. 6A-6B are front and rear perspective views of an embodiment of the disposable capsule assembly and dispensing device assembly as disclosed herein, awaiting for a pill to be dispensed.

FIGS. 7A-7B are front and rear perspective views of the disposable capsule assembly and dispensing device assembly shown disengaged, prior to the disposable capsule assembly being inserted into the dispensing device assembly.

FIGS. 8A-8C are front and rear perspective views of the disposable capsule assembly as disclosed herein.

FIG. 9 is aside perspective exploded view of the disposable capsule assembly of FIGS. 8A-8C.

FIGS. 10A-10B are front and rear perspective views of the dispensing device assembly as disclosed herein.

FIGS. 11A-11B are cross-sectional perspective views of the disposable capsule assembly locking and attaching to the dispensing device assembly as disclosed herein.

FIGS. 12A-12C are side and rear perspective sectional views of the disposable capsule assembly, showing the tamper resistant mechanism.

FIGS. 13A-13B are cross-sectional perspective views of the disposable capsule assembly, showing the mechanism of initiating the prescription for the user.

FIGS. 14A-14B are cross-sectional perspective views of the dispensing device, shown delivering and regulating the prescription to the patient.

FIGS. 15A-15C are cross-sectional perspective views of the disposable capsule assembly, showing the internal mechanism for delivering a pill.

### DETAILED DESCRIPTION OF THE INVENTION

It should be noted that the drawing numbers on subsequent views reference the same embodiment.

The description of this invention and its embodiments is not limited to a certain methodology, materials and modifications. The language used to detail the aspects of this invention are for the purpose of describing the aspects only, and should not be used to limit the claims made herein.

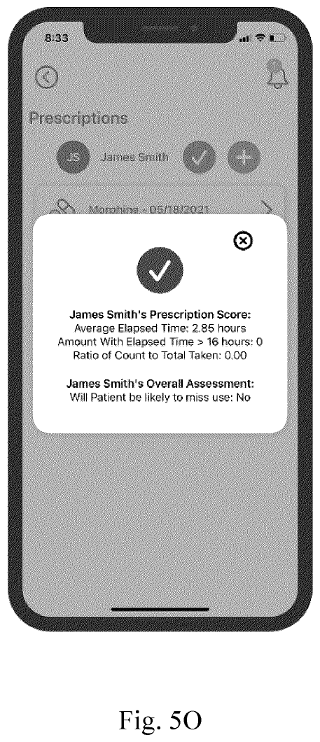

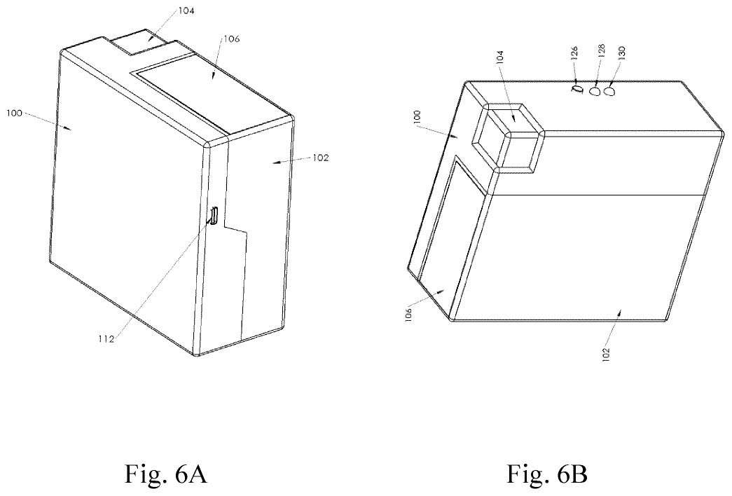

Referring first to FIGS. 6A-6B, a preferred embodiment of a live monitoring pill dispensing device 10 (sometimes referred to herein as “pill dispensing device”, “dispensing device” or “PillSafe Device”), which allows for a patient to take his/her prescribed medication, is shown. The dispensing device 10 comprises a dispensing device assembly 100 and a disposable capsule assembly 102. The disposable capsule assembly is configured to be inserted into, and attached to, the dispensing device assembly 100. The dispensing device assembly 100 is provided with a dispensing device button 104, which in this case is shown disposed at a top position on the dispensing device assembly 100, and indicates when a pill is ready to be taken/dispensed.

The medication (e.g. in the form of tablets or pills) is loaded into the disposable capsule assembly 102 by a medical professional (e.g. a pharmacist). A one-way locking lid 106 limits any later entry/access to the pills inside of the disposable capsule 102 by a patient.

An electronic circuit is recharged and sometimes directly powered through the power port 112. The uploading, checking connectivity, and unlocking actions of the dispensing device 10 are prompted by the use of an electronic push button 126, with a “successful” indication indicated, for example, using a green LED 128 and an “unsuccessful” indication shown using a red LED 130. Note that any form of electronic push button, LED screen, colored LED, or speaker may be used to indicate the successful or unsuccessful operations to the patient. The dispensing device assembly 100 and disposable capsule assembly 102 are preferably constructed using polypropylene plastic, but it should be appreciated that other materials may be used, such as other medical device approved plastics.

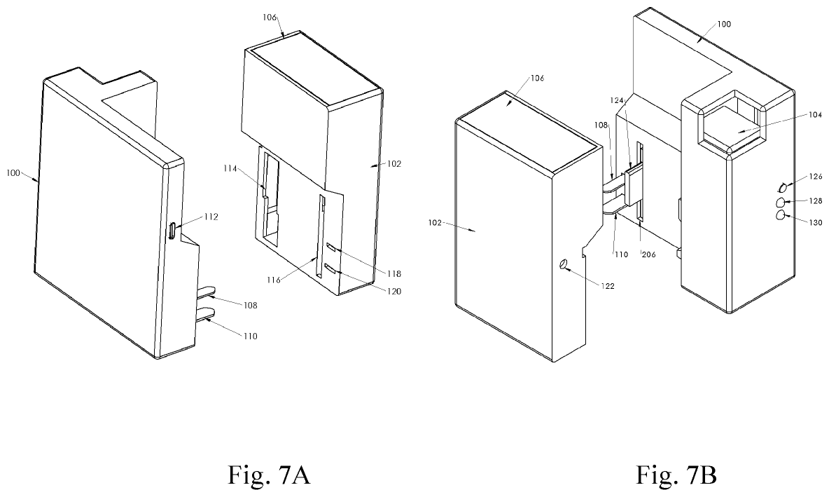

In FIG. 7A-7B, the live monitoring pill dispensing device 10 is not active, and the dispensing device assembly 100 is waiting for the disposable capsule 102 to be inserted therein and attached thereto. The superior component, the device button 104, includes four inferior components.

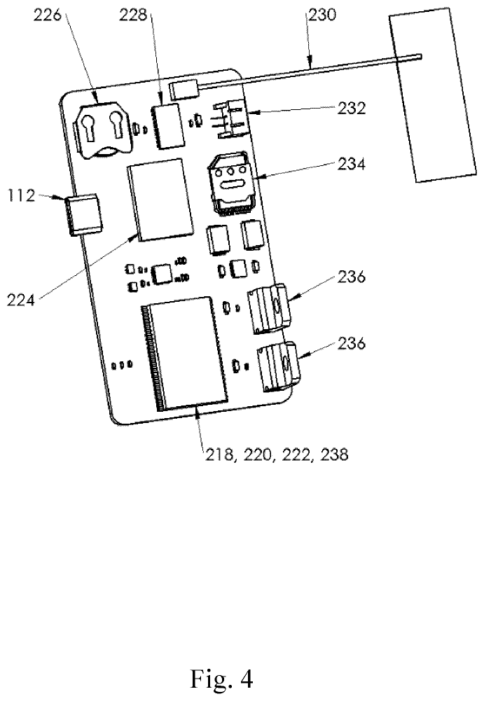

One of these inferior components is exposed in this view and is the agitator piece of the button 124. The agitator piece 124 of the button 104 is integrated with the button 104, and is guided by a vertical dispenser agitator hole 206 on the dispensing device assembly 100. The vertical dispenser agitator hole 206 is what restricts the dispensing button 104 to an up and down vertical motion. It can be observed that the dispensing button 104 is in the lower locked position to ensure that no pill can be dispensed until the disposable capsule assembly 102 is properly inserted into the dispensing device assembly 100. Electrically conductive prongs (upper prong 108 and lower prong 110) are used to ensure proper insertion orientation of the disposable capsule assembly 102 into the dispensing device assembly 100, and to regulate the delivery of power to the dispensing device assembly 100. Specifically, when the disposable capsule assembly 102 is fully inserted into and engaged with the dispensing device assembly 100, the upper prong 108 inserts into the disposable capsule assembly 102 through upper prong hole 118 and comes in contact with an electrically conductive material, wired to also contact the lower prong 110 which was simultaneously inserted through lower prong hole 120. The electronic circuit 178 more clearly shown in FIG. 4 will not receive any power from the main battery 176 unless the previously described prong circuit is complete. This is achieved by wiring the battery 176 to the upper prong 108, and the electronic circuit 178 to the lower prong 110. This ensures that only successfully loaded prescriptions turn on the dispensing device for use. To additionally ensure proper alignment of the disposable capsule assembly 102 into the dispensing device assembly 100, and to make sure the disposable capsule assembly 102 cannot be removed from the dispensing device 100 unless instructed by a medical professional, solenoid actuator 174 and its arm 182 are inserted into capsule locking hole 122 on the disposable capsule assembly 102.

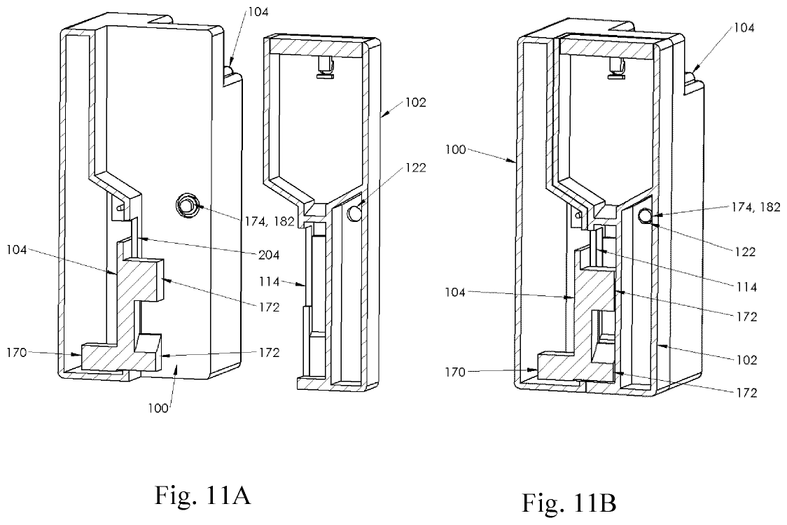

FIG. 11A-11B illustrates this locking process and shows how, when the disposable capsule assembly and dispensing device assembly are properly attached to one another, the solenoid arm 182 lines up with the capsule locking hole 122. Additionally, the lower locked position dispensing button 104 has inferior part 172 in line with the vertical guide hole 114 of the disposable capsule 102. Dispensing button inferior part 172 is what mechanically traps one pill/tablet, and what allows, when dispensing button 104 is pressed, for the single trapped pill/tablet to be dispensed. Just as the dispensing button agitator piece 124 of the dispensing button was guided by vertical dispenser agitator hole 206, the dispensing button inferior part 172 is additionally guided by a vertical hole 204 on the dispensing device assembly 100.

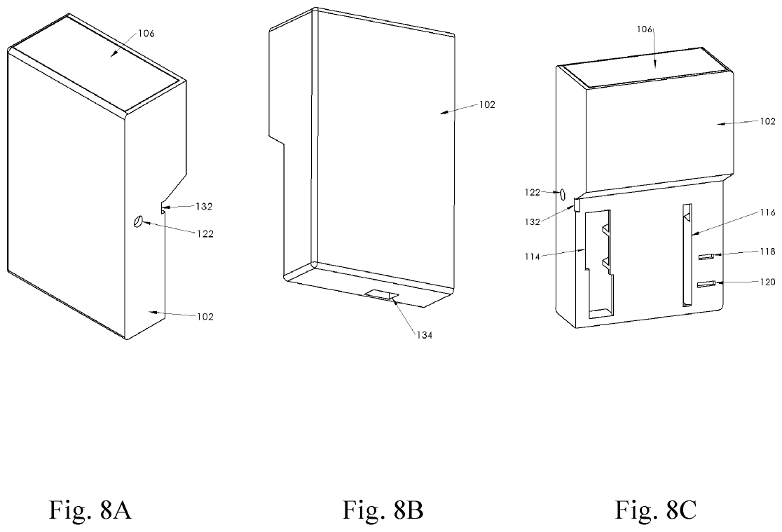

Further examining the disposable capsule assembly 102, FIGS. 8A-8C show the constructed disposable capsule assembly with a prescription inside that would be given to the patient for later use with the dispensing device assembly 100. Since the one-way locking lid 106 is properly positioned on the disposable capsule assembly 102, then the process of loading the pills has already taken place. On the bottom of the face of the disposable capsule assembly 102, the capsule dispensing hole 134 can be observed. This is where the pill exits after the dispensing button 104 is pressed. The front side of the disposable capsule assembly 102 shows where the vertical guide hole 114, agitator guide hole 116, upper prong hole 118, lower prong hole 120, and capsule locking hole 122 are located on the surface of the disposable capsule assembly 102. To ensure that the solenoid actuator arm 182 is pushed back before inserting into the capsule locking hole 122, a slanted edge 132 is used. All solenoid actuators used in this invention are pull solenoids with a returning spring. This is important as when slanted edge 132 pushes the solenoid arm 182 back, the spring forces the solenoid arm forward, and when the disposable capsule assembly is properly inserted, the forward arm will be pushed through the capsule locking hole 122.

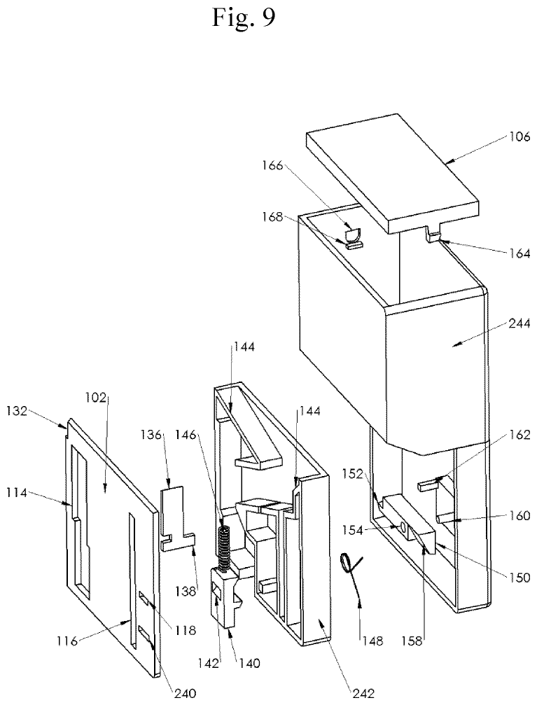

The disposable capsule assembly 102 further comprises mechanisms to ensure that the capsule is tamper resistant. FIG. 9 provides an exploded view of the various component parts of the disposable capsule assembly 102. 240 is the face of the disposable capsule that comes into contact with the dispensing device assembly 100. Disposable capsule face 240 has guide holes 114 and 116 for the dispensing button 104. Disposable capsule face 240 also has the slanted edge 132 for the locking solenoid actuator arm 182, and has prong holes 118 and 120.

In between the capsule face 240 and the capsule interior 242, tamper resistant shutter 140, tamper resistant shutter spring 146, and the trap door 136 are located. The tamper resistant shutter 140 and its corresponding spring 146 act to cover the prong holes 118 and 120 unless both prongs (108 and 110) are inserted properly. When prongs 108 and 110 are inserted through holes 118 and 120, the shutter 140 is forced upward and exposes a pathway 142 through the shutter for the prongs to continue. The shutter spring 146 acts to return the shutter and to deter tampering with the device easily. The trap door 136 has an arm 138 which protrudes through the capsule interior 242. When the disposable capsule assembly 102 is properly inserted and locked into the dispensing device assembly 100, then the trap door will fall, allowing for the pills to be taken. The capsule interior 242 is composed of tracks 144 which help guide the pills using gravity through to the disposable capsule assembly 102 and to the trap door 136. 244 is the main housing of the disposable capsule 102. The top of the housing is made to safely store the pills for the dispensing device 10. Along the top side of the housing 244, a hole 166 and stopper 168 are used to create the one-way locking effect with the one-way locking lid 106. On the sides of the one-way locking lid 106 are bendable wedges 164. When the one-way locking lid 106 is pushed into the capsule housing 244, the bendable wedges 164 flex until the hole 166 and stopper 168 are reached. Due to the bendable wedges' 164 shape, they are restricted from being pulled out of the hole 166 and also restricted from being pushed further into the capsule housing 244 by the stopper 168. The bottom of the capsule housing 244 has a pin 160 for the spinner 150. In between the capsule housing 244 and the capsule interior 242, the spinner 150 and the coil wire 148 are found. The capsule housing's pin 160 inserts into the spinner's hole 154. During manufacturing, either tear-away plastic or weak adhesive will be used to connect the bar 162 to the spinner 150. The coil wire 148 is what makes contact with the conductive prongs 108 and 110 to complete the power circuit previously discussed. As presently contemplated, a coil shape is used to produce a spring effect when the conductive prongs 108 and 110 are inserted, but any other shape, material, design may be used to complete the power circuit from the upper prong 108 to the lower prong 110.

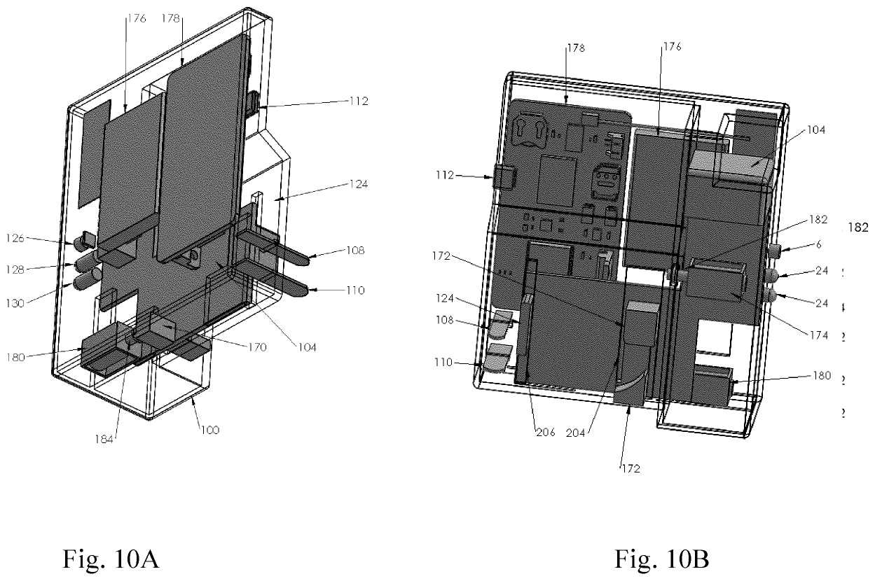

It should be appreciated that numerous components sit within the dispensing device assembly 100 as seen in FIG. 10A-10B. A first such component has been previously discussed and is the dispensing button 104. The dispensing button 104 is the superior component of inferior components such as the pill trapper 172, agitator 124, dispensing clip 170, and spring support 188. The dispensing clip 170 is the portion of the button that comes into contact with the dispensing solenoid 180 and its arm 184. The solenoid actuator 174 is orientated so that the arm 182 protrudes out of the dispensing device assembly 100. The electronic circuit 178 is positioned such that the recharging port 112 protrudes out of the dispensing device assembly 100. Power supply 176 may be a LiPo rechargeable battery, but could be any other battery or combination of multiple batteries that produce enough voltage and current to power the electronic circuit and its various components.

FIG. 4 illustrates a high level schematic view of the electronic circuit 178 used to control the dispensing device 10. The electronic circuit 178 is comprised of a microcontroller 218, a cellular module 224, a recharging port 112, a timer power supply 226, a timer 228, an antenna 230, a power supply connector 232, a sim card 234, a solenoid control 236, and a sensor 238. The microcontroller 218 has inferior components, processor 220 and memory 222, which store and execute computer language instructions. The memory 222 is additionally used to store prescription specific information, patient information, and database information. Prescription-specific information may include the date, time, number of pills taken, iterations, and the number of pills initially prescribed, etc. Patient information such as a uniquely generated identification number, date of the prescription on the dispensing device, and the name of the prescription may also be stored. The database information stored in memory 222 may include patient-specific token and database access keys. The date and time stored in the memory 222 reflect the instance that the pill becomes available for the patient to take and the instance that the pill is actually taken. This dual source of time data offers a further look into the patient's interaction with the prescription. The patient-specific information and database information stored in memory 222 are used to transfer all the saved prescription specific information to a database and categorically store it under the correct patient and prescription. Cellular module 224 in combination with sim card 220 get the device connected to 2G/3G/4G/5G cellular data networks in order to connect the dispensing device 10 to the database anywhere in the world. The microcontroller 218 has its own additional capability to communicate to the database or the user interface through classic Bluetooth™, Bluetooth Low Energy, or WIFI. A second power supply 226 is used to only power the timer 228. This feature ensures that the timer will always have enough power to keep the time regardless of whether the main power supply 176 is charged or not. The timer power supply 226 is designed to ensure that preferably the timer 228 can last the lifetime of the dispensing device without the need for recharging. Timer 228 is preferably a real-time clock with an integrated temperature compensated crystal oscillator and crystal. The timer 228 is connected to both the main power supply 176 and the timer power supply 226, which maintains accurate timekeeping even when the main power supply to the timer is interrupted. The real-time clock timer 228 maintains seconds, minutes, hours, day, date, month, and year information accurately. The solenoid control 236 uses the microcontroller 218 lower voltage control power to switch a gate allowing for the main power supply 176 to directly feed the solenoid actuators 174 and 180. Each solenoid actuator requires its own individual solenoid control 236 such that each can be separately controlled by microcontroller 218. In the present embodiment, the electronic circuit 178 is currently using the following: an Espressif ESP32 as the microcontroller 218; sim800L cellular module 224; USB-C recharging port 112; CR2032 timer power supply 226; DS3231 timer 228; GPRS antenna 230; 1.25 mm JST power supply connector 232; hologram nano sim card 234; TIP120 transistor solenoid control 236; and Espressif ESP32 touch sensor as sensor 238. Currently, the ESP32's built-in touch sensor is used to indicate when a pill is taken. The sensor 238 is set up beside the dispensing solenoid 180, such that when the solenoid arm 184 retracts, it comes into contact with sensor 238. The microcontroller 218 continually monitors the voltage of the main power supply 176. If the voltage drops past a predetermined level, then all functions but the sensor 238 are shut down to preserve the last bit of power. The predetermined voltage level is designed such that only the sensor 238 can be run for an extended period of time. This allows for the dispensing device 10 to capture any pills taken after the device is technically shut down to the patient. When the main power supply 176 is recharged, then normal operations may proceed. It should be appreciated that any other suitable microcontroller, timer, power supply, antenna, cellular module, or sensor may be used to store and execute the dispensing device's instructions.

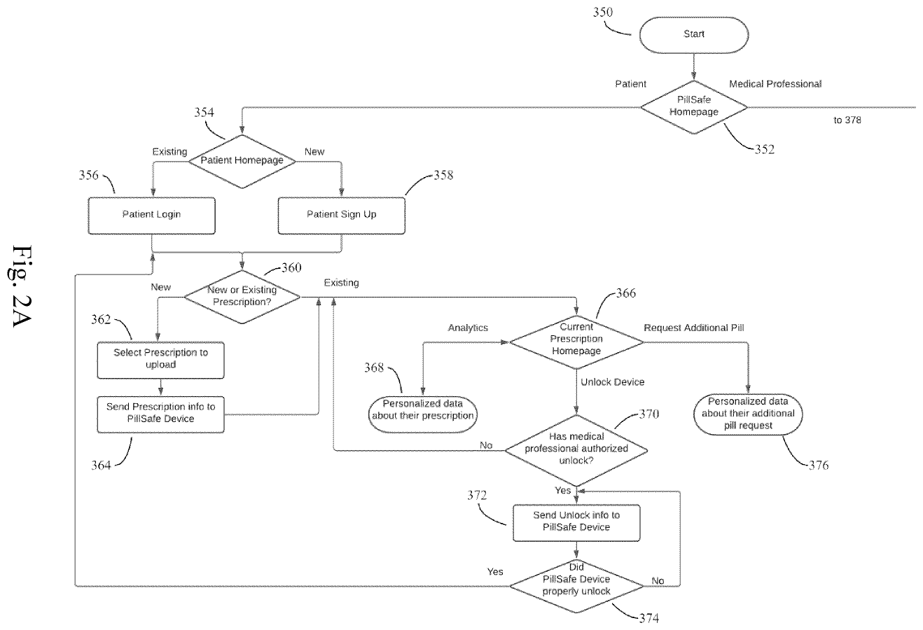

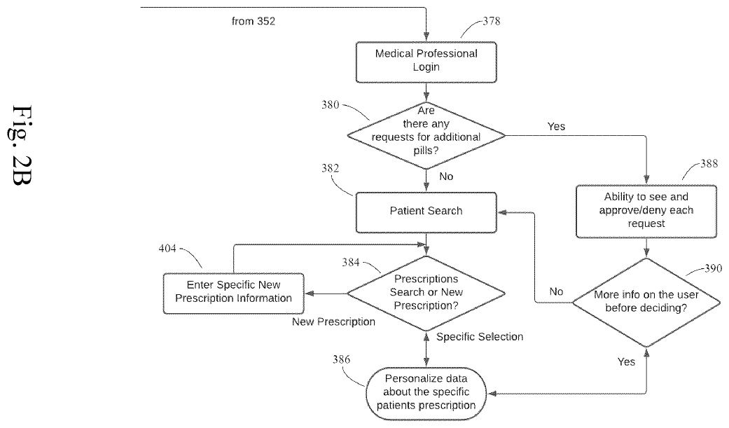

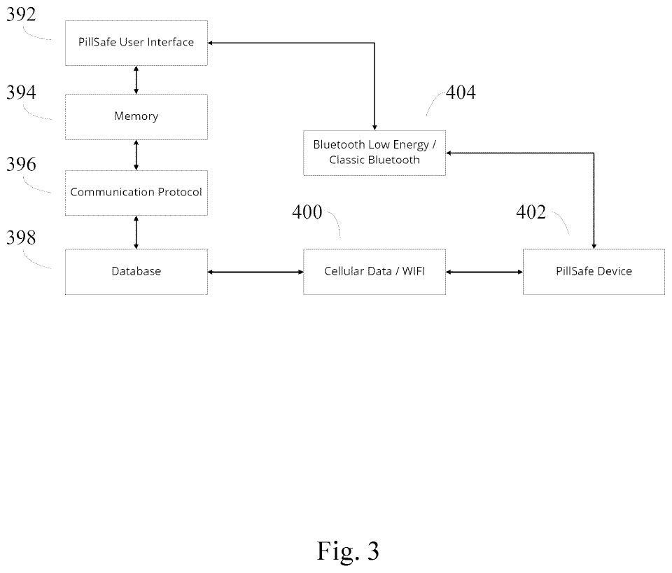

FIG. 3 further explains how the pill dispensing device 10 communicates to both the database and the user interface. Starting at PillSafe Device (step 402) can both send and receive data directly from the database 389 through the use of 2G/3G/4G/5G cellular data or WIFI (step 400). The PillSafe Device 402 can communicate directly to the PillSafe User Interface 392 through the use of classic Bluetooth or Bluetooth Low Energy. Every form of communication the PillSafe Device 402 has is encrypted for security of the data. The PillSafe User Interface 392 has its own memory 394 which helps limit the need for repeated sending and receiving of data from the database 389. The communication protocol 396 is the way that the data is sent or received from the memory 394 to the database 398. Currently, the preferred communication protocol 396 is using cellular data or 2G to reach the internet. The memory 394 also offers the ability for the user interface to still function while communication protocol 396 may be down.

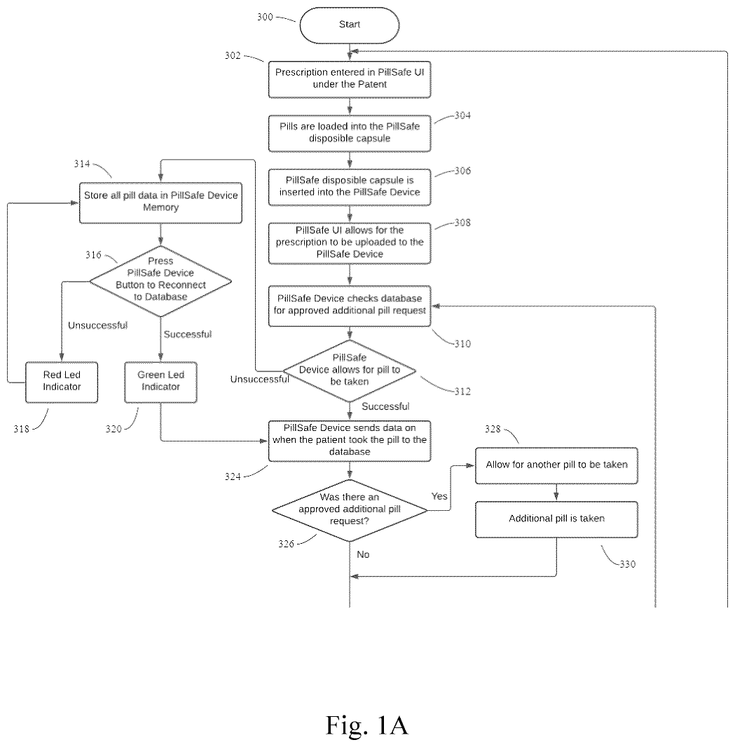

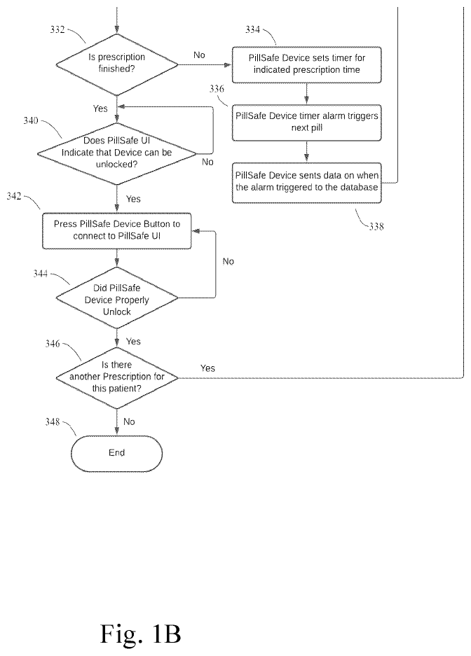

The following description is intended to illustrate one potential operation of the pill dispensing device 10 as described by the flowcharts found in FIGS. 1A-1B and 2A-2B. After a patient has been advised and prescribed by a medical professional to take an opioid prescription, the process may begin (step 300). The specific prescription needed by the patient (step 302) is entered into the PillSafe User Interface uniquely under the patient. In the PillSafe User Interface, this is done by selecting Medical Professional on the homepage (step 352) and entering the unique medical professional login information that was generated specifically for that particular medical professional (step 378). Searching for the specific patient (step 382) and selecting to enter a new prescription (step 384) allows for all the prescription details to be entered into the PillSafe User Interface (step 404). The prescription now exists in the system allowing the new patient the ability to sign up for the PillSafe User Interface's services. In the pharmacy, the specific pills needed for the prescription are poured/loaded into the disposable capsule assembly 102. The one-way locking lid 106 is put on top of the disposable capsule assembly 102, which signifies the prescription is loaded (step 304). Due to the pills being safely contained inside the disposable capsule assembly 102, it offers pharmacies the opportunity to both mail or distribute by hand knowing that they can monitor successful start and completion of the prescription.

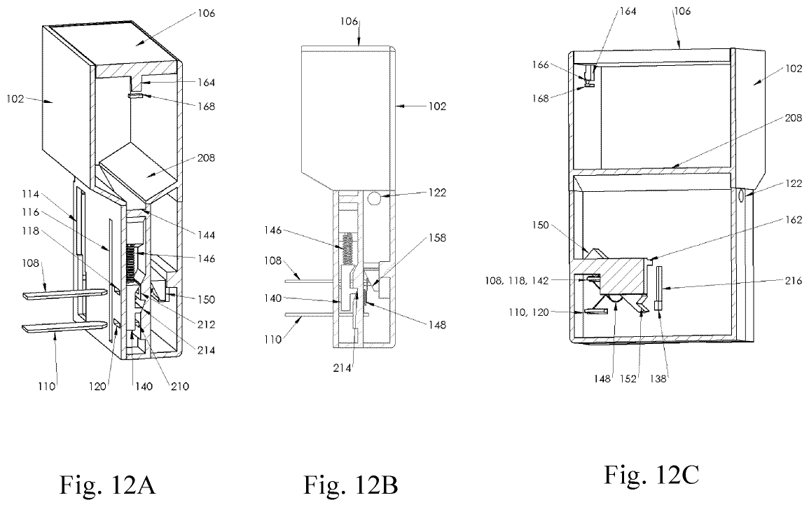

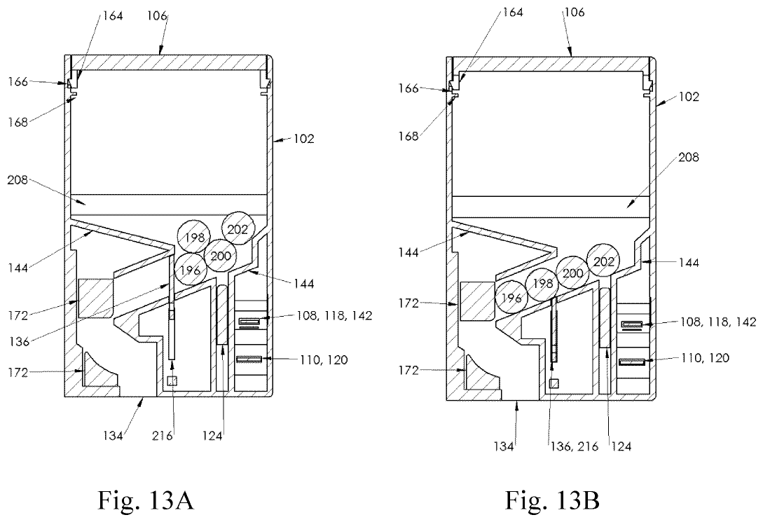

Once the patient receives the pills inside the disposable capsule assembly 102, the capsule assembly can be inserted into the dispensing device assembly 100 (step 306). This process of insertion bypasses the tamper resistant measures of the disposable capsule assembly 102, and allows only the dispensing device assembly 100 to access to the pills. Disposable capsule assembly 102 bypasses the tamper resistant measures when properly inserted into the dispensing device assembly 100 (see FIGS. 12A-12C and FIGS. 13A-13B). FIG. 12A shows how the disposable capsule assembly 102 is assembled for the patient. Such assembly will take place during manufacturing, except for the insertion of the one-way locking lid 106 into the disposable capsule assembly 102. The medical professional will fill the disposable capsule assembly 102 the same way a normal prescription is filled, e.g. by counting and pouring the pills using a funnel. Once the prescription is filled into the disposable capsule assembly 102, the medical professional may insert the one-way locking lid 106 into the disposable capsule assembly 102. At this point, the pills may only leave the disposable capsule assembly 102 when it is inserted into the dispensing device assembly 100. FIG. 12A depicts the instance right before the disposable capsule assembly 102 is inserted into the dispensing device assembly 100. At this point, the tamper resistant shutter 140 is forced down by the tamper resistant shutter spring 146, which makes the shutter 140 cover both prong holes 118 and 120.

The spinner 150 is held in place due to its physical connection with the disposable capsule assembly 102 using bar 162. The trap door 136 and its arm 138 are held at the top position since they are restricted by hole 216 of the capsule interior 242 through the arm 138 being held in place by the spinner 150 and its extended flat edge 152. In FIG. 12B, the dispensing device assembly's 100 prongs 108 and 110 enter the prong holes 118 and 120 of the disposable capsule assembly 102. Instantly, the tamper resistant shutter's angled side 214 comes into contact with the capsule interior 242 forcing the tamper resistant shutter 140 upwards. This compresses the tamper resistant spring 146, potentially causing the tamper resistant shutter to return to its original state if prongs 108 and 110 are removed. As prongs 108 and 110 move through and exit the disposable capsule assembly's interior through holes 210, both prongs come into contact with the coil wire 148. Using FIG. 13A to look inside the disposable capsule assembly 102, it can be observed that the trap door 136 is still blocking pills 196, 198, 200, and 202 from continuing down the tracks 144. At the instant of contact between the prongs 108 and 110 with the wire coil 148, contact with the spinner's slanted face 158 is also initiated (FIG. 12C). This contact between the upper prong 108 with the spinner 150 causes the spinner 150 to break free from the bar 162 of the disposable capsule assembly. Subsequently the spinner's extended flat edge will no longer hold the trap door arm 138 from falling down hole 216. FIG. 13B illustrates what occurs once the trap door 136 falls down, which allows the pills to continue down the tracks 144. As this trap door process occurs, the capsule locking hole 122 will simultaneously lock the disposable capsule assembly 102 to the dispensing device assembly's solenoid arm 182.

Even though the pills are now available to the dispensing device assembly 100, the patient still will not have access until this process is verified by the PillSafe User Interface. This verification process is done by uploading the data about the prescription from the PillSafe User Interface to the PillSafe Device, for example through the use of Bluetooth Low Energy. The PillSafe User Interface ensures that the disposable capsule assembly 102 is properly inserted into the dispensing device assembly 100 because the electronic circuit 178 will not receive power from the power supply 176 otherwise. This is through the use of prongs 108 and 110 and the use of the coil wire 148 to complete the circuit. The patient initiates the upload process (step 308) by hitting the electronic push button 126 on the dispensing device assembly 100. Concurrently, the patient navigates from the PillSafe Homepage 352 to the Patient Homepage 354, where they can sign up (step 358) or login (step 356) to the system depending on their status in the system. The backend code of the User Interface recognizes that there is no active prescription (step 360) and prompts the patient to select the prescription on the device (step 362). This may be verified using bar codes or Quick Response (QR) codes and object detection. The dispensing device assembly 100 has its own unique identification number assigned to the QR code sticker placed on the device. The disposable capsule assembly 102 also has a unique combination of patient and prescription information assigned to the QR code sticker placed on the capsule. Through the use of a camera in conjunction with the User Interface, both QR codes are simultaneously scanned while also observing for the object representing the PillSafe device. This allows for the User Interface to detect if the proper disposable capsule assembly is inserted into the dispensing device assembly. In order for the object detection to recognize that the disposable capsule assembly 102 is inserted into the dispensing device assembly 100, a machine learning model is trained. Images are first taken of the dispensing device assembly 100, disposable capsule assembly 102, and then the two locked together. The images are then labeled and classified depending on which of the three situations are depicted. For example, if the image is showing the disposable capsule inserted into the dispensing device, then the image would be classified as locked together and labeled accordingly. Once all the images have been classified and labeled, a neural network object detection model nay be trained to recognize when either the dispensing device, disposable capsule, and the two locked together is shown using the camera and the User Interface. Using this trained model, the User Interface can use the object detection to verify if a dispensing device is locked to the disposable capsule. While the object detection model is confident that the two assemblies are locked together, a QR code on the dispensing device assembly 100 and a QR code on the disposable capsule assembly 102 are simultaneously read. These QR codes in conjunction with the object detection may be used to confirm that the correct disposable capsule holding the correct prescription is locked to the correct dispensing device. After successful verification of the correct prescription, the prescription information is sent via Bluetooth Low Energy to the PillSafe Device (step 364). Upon reception of the prescription information, the PillSafe Device reads the database for the first time, gaining all the necessary information to begin the distribution of the pills to the patient.

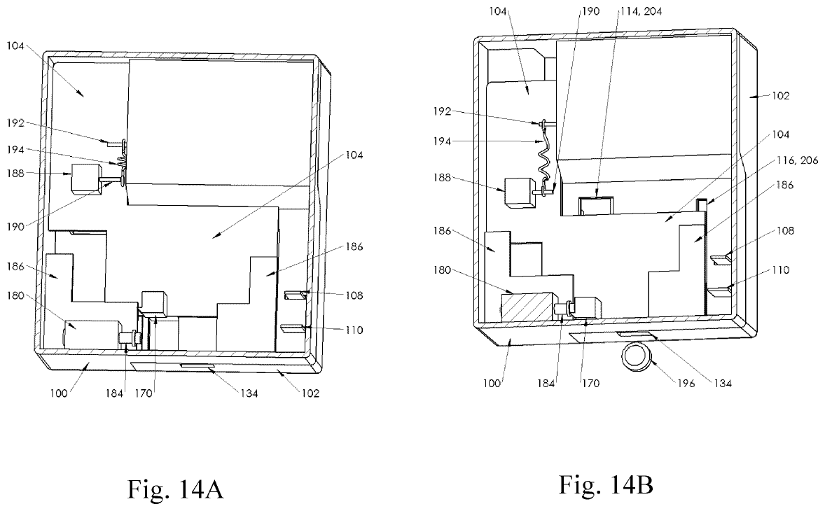

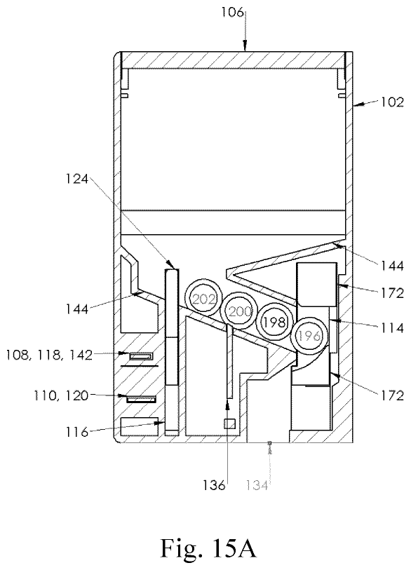

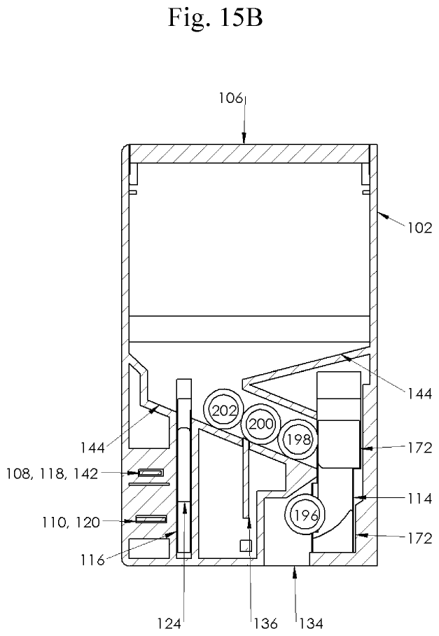

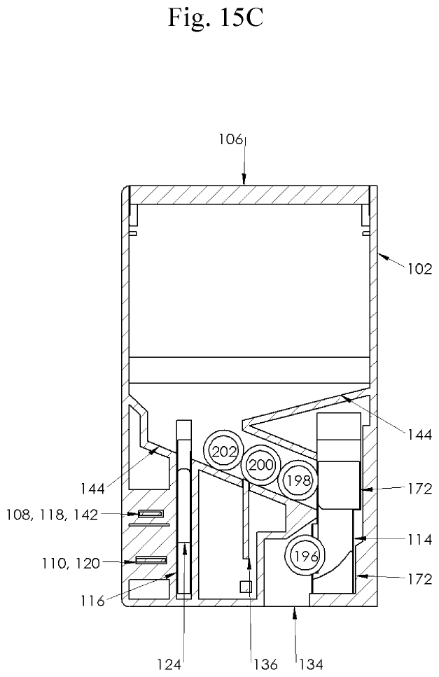

Up till this point, dispensing button 104 is locked at the bottom position due to the dispensing clip 170 being held down by the dispensing solenoid 180 and its arm 184, as seen in FIG. 10A. Now that the PillSafe Device is ready to begin distribution of the pills, solenoid 180 is powered and its arm 184 is withdrawn from restricting the dispensing clip's 170 movement. The dispensing button 104 is mechanically connected to the dispensing device 100 through the use of pins 190 and 192, connector 188, and button spring 194. Now that the button dispensing clip 170 is free to move, the button spring 194 contracts and returns the dispensing button 104 to the top position. As this is occurring, the PillSafe Device may check the database for approved additional pill requests (step 310). In this illustrated instance, none is requested and the patient is now able to take the pill as illustrated by FIGS. 14A-14B and 15A-15C. FIG. 15A shows the interior of the PillSafe Device before a pill has been allowed to be dispensed/taken. FIG. 15B shows the interior of the PillSafe Device right after the solenoid has engaged and the spring has returned the dispensing button to the top position. Between the two positions, the pill trapper 172 goes from holding back pill 196 to then trapping it within the gap of the pill trapper 172. FIG. 14A shows a cutaway of the dispensing device assembly 100 as the spring 194 holds the button 104 in the top position. The time in which the button is released and allowed to reach the top position is referred to as the alarm time and is saved to memory 222. When the patient is ready to take the pill, the dispensing button 104 is physically pressed down. FIG. 14B illustrates pill 196 falling out of the dispensing hole 134, with spring 194 stretched and extended, and solenoid 180 and its arm 184 restricting the button clip 170 and the button 104 from returning to the top position. FIG. 15C illustrates how pill 196 is trapped in the pill trapper 172, and how when the button 104 is pressed, pill 196 is free to fall out of the dispensing hole 134. When solenoid 180 first holds the dispensing button 104 in the lower position, the time is saved to memory 222 as the time the pill was taken.

The success of a pill being taken is decided by the ability for the PillSafe Device to connect to the database (step 312). If a connection is not made, then it was unsuccessful and all pill data continues to be saved on the memory of the PillSafe Device (step 314). The PillSafe Device will not allow for any more pills to be taken until connectivity to the database is re-established. Connectivity is established by the patient hitting the electronic push button 126 any time after a prescription has been uploaded to the dispensing device assembly 100. If the connection is still unsuccessful, then the red LED 130 will illuminate (step 318) and the patient will have to try again before another pill is allowed. If the reconnection to the database was successful (step 320), then the green LED 128 will illuminate and standard database protocol will resume.

If connectivity to the database is established after taking the pill, then the PillSafe Device sends the alarm time and taken time data to the database (step 324). At this point, if an additional pill request was approved and flagged by the device, it would then prompt the patient to take another pill (step 330). This would occur again by solenoid 180 unrestricting the movement of the dispensing button 104 from returning to the top position, and the patient then physically pressing the dispensing button back down. The PillSafe Device then calculates if the prescription is finished (step 332), which at this time is not true as only the first pill was taken. At this point, the timer 228 will schedule an alarm to trigger after the allotted time specified/requested by the medical professional between each dosage has passed (step 334). After the allotted time passes and the alarm goes off (step 336), the PillSafe Device sends the alarm time to the database (step 338). If it is unsuccessful, it will still allow the pill to be taken but it will try again later with the taken time as previously discussed. From this point on, the PillSafe Device will repeat the steps of the solenoid 180 unrestricting the dispensing button 104 from returning to the top position, the patient forcing the dispensing button 104 back to the bottom position and releasing the pill 196, recording the times of the alarm and the taken pill, sending the data to the database, connecting to the database if needed, and administering additional pills until all the pills in the prescription are taken and the prescription is finished (steps 312-332).

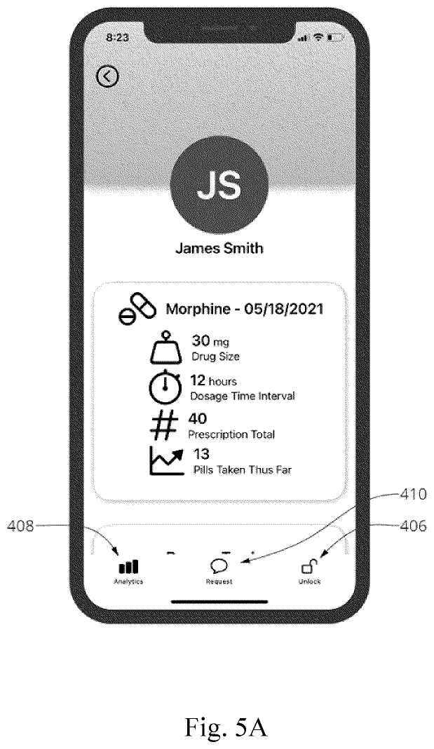

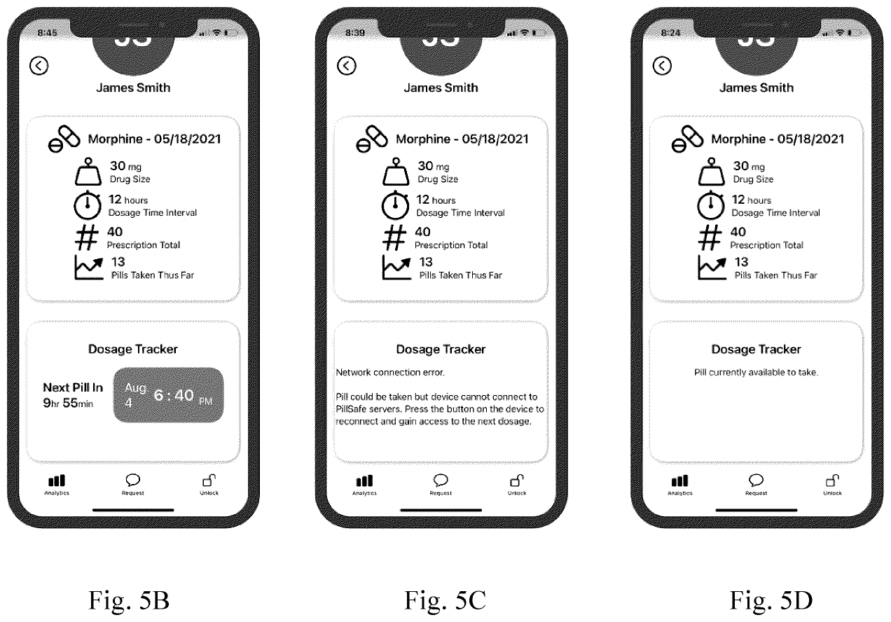

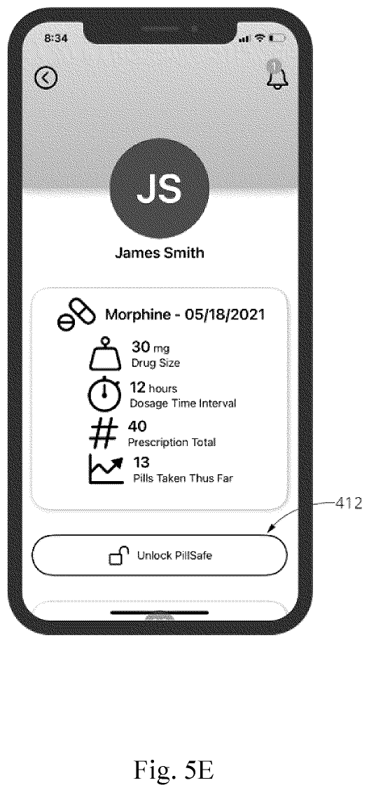

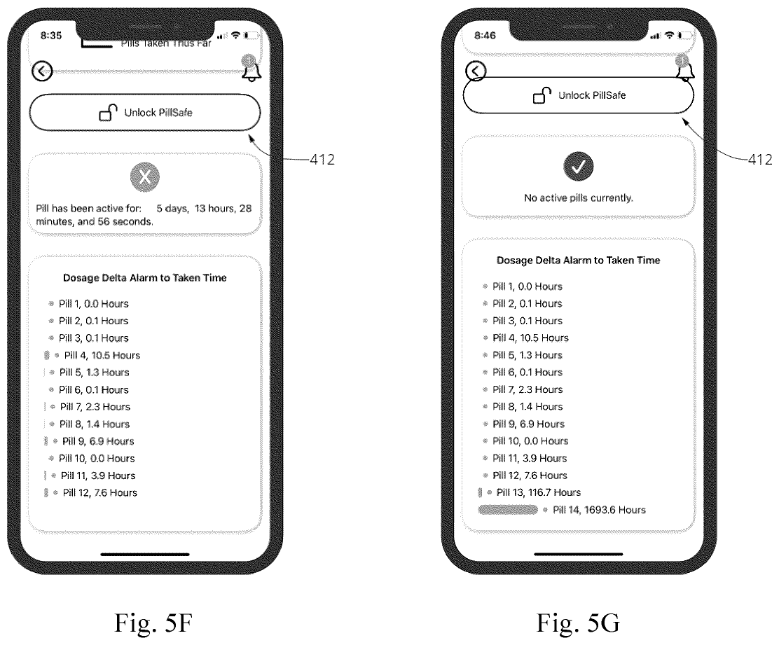

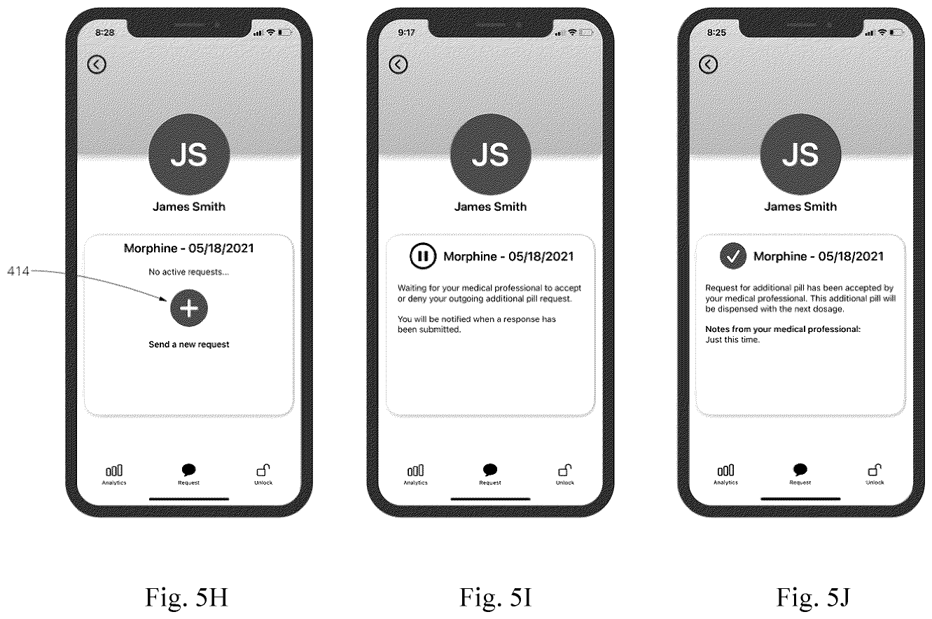

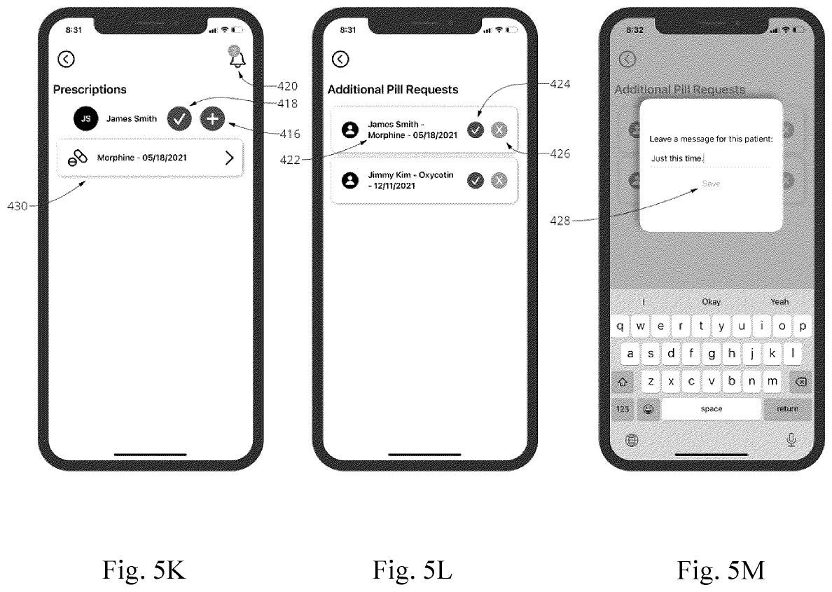

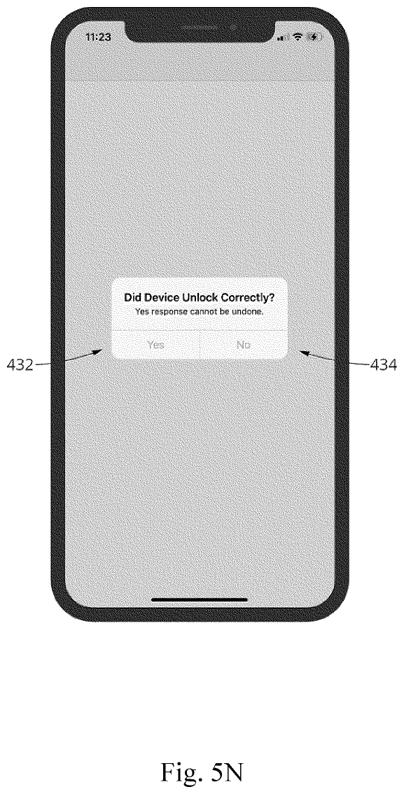

Throughout the process of the pills being taken from the PillSafe Device, both the patient and the medical professional have a variety of analytics at their disposal within the PillSafe Device User Interface. Because the PillSafe Device has the capability to access the database remotely and independently, it offers up-to-date or live data to be shown to both the patient and the medical professional. Resuming from where the patient has successfully logged in (step 356) and the backend code recognises that there is an existing or active prescription on the PillSafe Device, then a screen like FIG. 5A (step 366) gives the patient the ability to see the current prescription analytics (steps 408 and 368). FIGS. 5B-5D show three options of what the backend code could apply to the situation. All three options share the same basic prescription information such as the type, name, start date, total number of pills, pills taken so far, and the pills left in the prescription. What can vary are the diagnostics of the PillSafe Device. For example, FIG. 5B shows what a patient would see if the dispensing button 104 was restricted in the bottom position. The information shown details the exact time, date, and elapsed time from current time until the alarm will trigger and the dispensing button 104 will be released. Another potential diagnostic seen in FIG. 5C is telling the patient that their device couldn't connect to the database, and that the electronic push button 126 needs to be pressed in order to re-establish the connection. FIG. 5D shows the last diagnostic option potentially being shown to the patient. This diagnostic is letting the patient know that the dispensing button 104 is actively at the top position waiting for the patient to press it down and administer a pill. Thus, a pill is currently available to the PillSafe Device to be taken by the patient.

Turning now to where the medical professional successfully logged in and searched the patient, they can now search for the various prescriptions under the selected patient as illustrated in FIG. 5K. Step 430 is an example of one prescription for this patient and this section shows where each prescribed prescription would populate showing the type and date of the prescription. Selecting the check patient button (step 418), the medical profession gets a quick snapshot of the selected patient's overall prescription score and risk assessment as illustrated in FIG. 50. The overall prescription score may be established by taking average statistics over all prescriptions under the selected patient. The risk assessment addresses how risky this patient is with respect to being prescribed opioids and it can be used to predict or assesses the likelihood of future abuse. Such risk assessment may be calculated based on certain parameters, minor calculations and/or human input, but it is contemplated that future applications may also utilise artificial intelligence and machine learning to make a similar assessment. The following is one example of how artificial intelligence and machine learning may be used to calculate the risk assessment of a patient. Other predictions of patient adherence can be made but the process will be the same. All that changes for other risk predictions is that a new machine learning model would need to be trained for each desired risk prediction output. One risk prediction output based on the PillSafe devices captured data is to determine whether or not a patient is going to finish the prescription. Input data such as the number of pills taken, number of pills in the prescription, and amount of time between the alarm and taken time for each pill may be formatted into column tabular data format. The output data will be a “yes” or “no” response based on if the prescription is finished. The finishing of a prescription is measured by the successful unlocking of the disposable capsule from the dispensing device. Using a select number of patients' past data regarding their relationship with finishing their prescriptions, a neural network regression model may be trained. This model will then be used to assess whether new or active prescriptions run the risk of the output being a “no”. This means that the model is predicting with a certain confidence that the patient in question will/will not finish the current prescription. To further examine specific prescription data (step 386) instead of all the prescription data at once, the searched prescription (step 430) may be selected. FIGS. 5E-5G are the data for the specific selected prescription (step 430). FIG. 5E is the basic information, such as patient name, prescription start date, drug name, drug type, time between each dosage, number of pills prescribed, and how many pills have currently been taken. Unlock PillSafe Button (step 412) triggers the ability for the patient to unlock the dispensing body assembly 100 from the disposable capsule assembly 102.

FIGS. 5F-5G represent four potential circumstances that the bottom part of this searched prescription 430 data. First, FIG. 5F may present a graph or chart depicting a patient that is not abusing based on the elapsed time between the alarm and take time being less than a calculated “abuse time”. The applicable abuse time for a particular prescription may be determined for each particular prescription, e.g. currently set up to be sixteen hours (the suitable abuse time may be determined/refined through additional clinical trials). When the elapsed alarm to take time is less than the abuse time, then the data is shown in green; whereas, when greater than the abuse time, the data is shown in red. Because each pill on the y-axis of the graph does not have an elapsed alarm to take time greater than the abuse time, then the medical professional can deduce that the prescription is being correctly followed. The wording above the graph tells the medical profession if there is a pill currently available for the patient. The second scenario can be seen here where the text has an elapsed time from when the last alarm went off on the device to the current time. Scenario three can be seen in FIG. 5G, where pill 13 and 14 in the graph had an alarm to take time greater than the determined threshold. This lets the medical professional visually see that this patient may be abusing if this trend continues. The fourth scenario as seen in the text above, tells the medical professional that there is no pill currently available for the patient to take. This would indicate to the medical professional that the prescription is currently being followed and that the patient is not currently allowed to take a pill. The graph will represent many different pill taking scenarios, but will only show which ones had longer alarm to take times, while the text above the graph will either tell the elapsed time from the alarm to the current time or that no pill is currently available. If a patient is abusing a prescription, the medical professional would be able to see it in this screen of the user interface.

While the prescription is still active on the PillSafe Device, the patient has the ability to request additional pills to be taken at the time of the next dosage. As seen in the device flow operations (FIG. 1A-1B), with each pill taken the PillSafe Device checks for an approved additional pill request 310 before potentially distributing the additional pill (steps 326, 328, and 330). Resuming again from where the patient has successfully logged in (step 356) and the backend code recognises that there is an existing or active prescription on the PillSafe Device, then a screen like FIG. 5A 366 gives the patient the ability to see the request for additional pills button 410 and 376. If the button is selected and no other requests for additional pills have been made, then FIG. 5H is what will give the patient the ability to create a new request 414. It must be appreciated that currently the additional pill request feature is designed just to allow a single pill to be added at the time the next pill on the device is ready for the patient. This current design should not limit the terms of this invention, as it is contemplated that the device can also be switched to have multiple pills requested and dispensed at the exact time it is approved by the medical professional. FIG. 51 is the user interface screen shown to the patient right after the create a new request button 414 was hit. This screen indicates to the patient that the most recent request for additional pills is still pending a response from their designated medical professional. Once responded to by the medical professional, FIG. 5J shows the response to the patient. This screen illustrates to the patient if the request has been accepted or denied, when to expect the additional pill, and any specific note mentioned by the medical professional. Because this screen is representing an accepted request, then the device would be flagged at the next dosage saying that an additional pill was approved 326, and that it can be taken by the patent at this time (steps 328 and 330). It must also be appreciated that only one additional pill request can be made for each pill in the dosage. This means that the medical professional could potentially receive one request for every pill in the prescription. It is also contemplated that the user interface can be configured to allow the medical professional to predetermine and pre-approve a select number of additional pill requests at once.

Turning again to where the medical professional successfully logged in and searched the patient, the medical professional can now see they have a notification 420 as illustrated in FIG. 5K. The notification button 420 is available on all user interface screens 382 and 384. This allows for the notifications to be checked throughout the use of the user interface. The notification screen in FIG. 5K may be one of many. When the notification button 420 is selected, FIG. 5L is presented to the medical professional. This user interface screen will list out every request for additional pills made by a patient entered by the medical professional. It must be appreciated that currently only the medical professional who entered the prescription has access to approve additional pill requests, but future plans are to expand this to allow for the medical professional to select other medical professionals to have the capability for the specific patient. Additionally, the current design allows for the medical professional to turn off the ability for a patient to request additional pills. This could be advantageous for patients who show signs of potential abuse, or if the prescription does not call for the need. Each line represents an active additional pill request that the current medical professional needs to address 380. Each line will include the name of the patient, name of the prescription and a “yes” or a “no” 388. If the medical professional wants more information to inform their decision on the additional pill request (step 390), then the patient's name button may be selected. This will take the medical professional to the previously described user interface screen as illustrated by FIGS. 5E-5G. This opens the specific prescription that is being requested analytics for the medical professional to use for their decision on the additional pill request. If the back button is pressed, the medical professional returns back to FIG. 5L, where they can input their informed decision. Selection either “yes” 424 or “no” 426 will either accept or deny this specific request for additional pills. When either choice is made, FIG. 5M prompts the medical professional to leave a message for the patient describing their choice. When the save button 428 is selected the response is sent to the patient and the request is no longer active.

When a prescription is finally finished, the disposable capsule assembly 102 remains locked to the dispensing device assembly 100 until instructed by a medical professional. As previously discussed, when the medical profession hits the unlock PillSafe button 412 on their user interface screen as seen in FIG. 5E, then the patient now has the ability to unlock their disposable capsule assembly 102 from the dispensing device assembly 100. Resuming again from where the patient has successfully logged in (step 356) and the backend code recognises that there is an existing or active prescription on the PillSafe Device, then a screen like FIG. 5A 366 gives the patient the ability to see the unlock button 406 and 370. It must be appreciated that the unlock PillSafe button 406 stays hidden from the patient until the medical professional hits the unlock PillSafe button 412. When the patient hits the unlock PillSafe Device button 406, then the patient needs to initiate the unlock process 342 by hitting the electronic push button 126 on the dispensing device assembly 100. Through the use of Bluetooth Low Energy, the device is sent information confirming that the medical professional has issued for the dispensing device to be unlocked. Upon successful confirmation, locking solenoid 174 activates and its arm 182 retracts. As described previously for uploading a prescription to the PIllSafe Device, successful confirmation is achieved when the object detection recognizes that the disposable capsule assembly 102 is locked to the dispensing assembly 100 and when the correct QR codes are simultaneously read. This allows for the disposable capsule assembly 102 to be removed from the dispensing device 100. At this point, the disposable capsule 102 can be thrown out or recycled. At this point in the user interface, no new prescriptions can be started till the patient accepts full responsibility for successfully unlocking the PillSafe Device as seen in FIG. 5N. If the device did not unlock properly (step 344), then the patient will hit the “no” button 434. This will allow the patient to repeat the Bluetooth process until the patient indicates that the device has been properly disconnected. Once the patient indicates that the device has been properly unlocked by hitting the “yes” button 432, then the patient is allowed to add a new prescription to the dispensing device 10 and repeat the whole process over again with a new prescription loaded. The dispensing device assembly 100 is designed to never have to be changed out, and allow for continued prescriptions to be taken through the use of multiple disposable capsule assembly 102 being used. It must be further appreciated that the Bluetooth Low Energy transmission used for both the upload and unlock features of the PillSafe Device implements the use of a list of available devices. When the patient hits the electronic push button 126, the device will appear in the device list on the user interface. When a PillSafe Device is selected from this list, the backend code checks that this is actually the right device for the patient's actions. If the wrong device is selected then the patient will have to repeat the process. If not, then the process goes as normal, and the User Interface then prompts the use of the camera for the object detection and QR code verification process. Additionally, all communication protocols discussed will implement the use of encryption for cyber security purposes.

It is also contemplated that either the inside, outside, or the plastic itself of the PillSafe Device will be made from or covered in an electrically conductive material. Such material includes, but is not limited to, electrically conductive tape, paint, or metal. One embodiment of such design would be where the inside of the disposable capsule assembly 102 that holds the pills is painted with an electrically conductive paint. The microcontroller in the electronic circuit of the dispensing device would be physically wired to the conductive paint such that the microcontroller can send an electrical current through the paint. If the disposable capsule assembly 102 is broken into, then the conductive paint would change electrical properties. Such properties could include but not limited to voltage, resistance, current, or power. All described electrical properties would be monitored by the microcontroller. Physical breaking the disposable capsule assembly 102 will change the electrical properties sent through the paint, which when the properties drop below a pre-determined threshold will tell the microcontroller that the device has been broken into.

It is contemplated that some features of the present device, system and method include, without limitation, one or more of the following:
A system and method for managing a patient's pill description through the use of a dispensing device comprising a dispensing device assembly and a disposable capsule assembly, wherein the dispensing device is configured to communicate (e.g. via data or wireless networks) with a patient prescription management database.
The dispensing device assembly is provided with an electronic circuit comprising: (i) a microcontroller with memory; (ii) a timer; (iii) an individual power supply for the timer; (iv) an individual power supply for the microcontroller; (v) a dispensing button for dispensing the pills; (vi) a solenoid actuator configured to lock and unlock the dispensing button; and (vii) a sensor for detecting when a pill has been dispensed and/or when the button has been pressed.
The dispensing device assembly includes a button which, when pressed, dispenses a single pill (or optionally a single dose of pills, in the case where a patient's prescription dosage may be of multiple pills) to the patient through an outlet provided by the dispensing device assembly.
The available operations of the dispensing device may be stored in the memory of the dispensing device. Prescription data (e.g. such as when a pill should be taken, how frequently, how many pills in a dose, how many pills in a total prescription, name of prescribed drug, name of patient, etc.) may also be stored in the memory.
Dispensing device data (such as the time a pill was allowed to be taken and the time it was actually taken, name of patient, etc.) are stored in the memory of the dispensing device. Such dispensing device data may be stored and retained even when power is lost.
The dispensing device is configured with communication capabilities to allow separate direct communication to the database and any user interface (e.g. of patient or physician).
The dispensing device assembly is adapted to restrict the ability of a patient to take their next pill (until a certain time, time period or other condition) by locking the button in place.
The dispensing device assembly, when triggered from the timer, has the ability to release the button and allow the patient to press the button for a pill.
The dispensing device assembly may be provided with a sensor(s) for tracking/logging the release and pressing of the button and communicating such information to the microcontroller.
The dispensing device assembly only turns on power when properly connected to the disposable capsule assembly.
The dispensing device maintains dual power sources to ensure that operation power is separate from the time-keeping power, resulting in a timing device that is always accurate and available.
The dispensing device maintains sensor capability while shutting power off to the patient when battery power is running low.
The dispensing device may be configured to only operate when the correct disposable capsule is inserted.
The dispensing device may be configured to require a patient to scan QR codes and the PillSafe device through the camera, in order to properly verify that the correct prescription has been loaded to the dispensing device
The dispensing device assembly does not allow for disposable capsule assembly to be removed during normal operation.
The dispensing device allows for the disposable capsule assembly to be removed when approved by the patient's physician, indicated by the user interface, and verified through the object detection and QR codes.
The disposable capsule assembly provides a method for storing all the pills in the prescription without changing how pharmacists normally fill prescriptions.
The disposable capsule assembly can provide one-way locking storage such that when the lid is secured on top, no other entry/access to the pills is possible, other than when the disposable capsule assembly is inserted/installed in the dispensing device assembly.
The disposable capsule assembly is configured to allow a path for electrical power to return to the dispensing device assembly.
The disposable capsule assembly provides a tamper resistant guard such that only when the capsule assembly is inserted into the dispensing device, do the pills become accessible.
The purpose of the disposable capsule assembly is to hold the prescription, whereas the dispensing device assembly can be configured to reload and hold later filled disposable capsules assemblies.
The disposable capsule assembly provides a means to electronically notify the dispensing device assembly in the event a patient breaks into the disposable capsule assembly (or in the event that the integrity of the disposable capsule assembly is otherwise compromised).
The disposable capsule assembly offers human to machine interactions such that the patient is notified when issues arise in the dispensing device.
The disposable capsule assembly's shape offers a gravity-fed path for a single pill to be arranged for the button of the dispensing device assembly to offer up to the patient.
The dispensing device and system may be configured to allow the physician through a user interface to permit the dispensing button to be pressed multiple times.
The user interface can also be configured to allow a patient to make a single request to the physician for an additional pill(s), between each dosage, following which the physician is notified via the user interface. User interface interactions may be saved to the database, so that the dispensing device can be notified when changes/responses to the patient request occur.
A physicians will be provided with the ability to accept or deny additional pill requests. A physician may also be provided with the ability to turn off additional pill requests for a patient(s).
The time that a pill was allowed to be taken and the time the pill was actually dispensed can be logged and communicated to the database. The difference in these times can be tracked as an average of as a total, and displayed/communicated to a physician as necessary and stored to the database to provide a general overview of how the patient adheres to a prescription(s).
Dispensing device data for all patients in the database can be analysed to find trends/comparisons and used by the system to teach itself (using machine learning techniques) to predict whether or when patients are likely to misuse.
The system may be configured to analyze the dispensing device data in order to notify a physician through the user interface if a current patient is abusing a prescription.
It is contemplated that the system can be configured to determine that the correct disposable capsule assembly and its accompanied prescription is inserted into the correct dispensing device assembly, by scanning a barcode or QR code and simultaneously recognizing the shape of the PillSafe Device through machine learning
The dispensing device may be made from pharmaceutical approved FDA plastics such as polyethylene.

1. A pill dispensing device for managing and tracking the dispensing of pills to a patient, the pill dispensing device comprising:
a pill dispensing assembly; and
a tamper-resistant disposable capsule assembly,
wherein the disposable capsule assembly and the pill dispensing assembly are configured to engage with each other in an engaged configuration,
wherein the pill dispensing assembly comprises:
a dispensing button for dispensing the pills;
an electronic circuit comprising: (i) a microcontroller with memory; (ii) a first power supply for powering the microcontroller; (iii) a timer; (iv) a solenoid actuator configured to lock or unlock the dispensing button; (v) a sensor configured to detect when the dispensing button has been pressed and when a pill has been dispensed; and
(vi) communication means; and
a patient user interface configured to receive input from a patient and configured to communicate prescription information to the patient;
wherein the microcontroller is configured to store dispensing device data in the memory;
wherein the microcontroller is configured to actuate the solenoid actuator according to the dispensing device data;
wherein the microcontroller is configured to communicate the dispensing device data via the communication means to an external patient database;
and wherein the disposable capsule assembly comprises: a housing for containing a plurality of pills; a locking lid; a trap door configured to open in response to the patient pressing the dispensing button, thereby allowing one or more of the plurality of pills to be dispensed from the dispensing device to the patient.

2. The pill dispensing device of claim 1, wherein the dispensing device data includes one or more of: prescription data, patient data, time from the timer, name of prescribed drug, amount of pills in a total prescription, prescription dosage (number of pills in a dose), frequency of dispensing each dose, time period between respective doses, prescription frequency, date and time of prescription activation, time/date when each dose is made available to the patient, time/date when a dose is dispensed to the patient, expiry of prescription, and time for renewing a prescription,

3. The pill dispensing device of claim 2, wherein the microcontroller is configured to actuate the solenoid actuator and unlock the dispensing button according to the time/date when each dose is to be made available to the patient.

4. The pill dispensing device of claim 3, wherein the dispensing deice is configured to lock the dispensing button, once a pill or a dose of pills have been dispensed.

5. The pill dispensing device of claim 1, wherein the pills are opioids.

6. The pill dispensing device of claim 1, wherein the electronic circuit additionally comprises a second power supply for powering the timer.

7. The pill dispensing device of claim 1, wherein power to operate the microcontroller is supplied only when the disposable capsule assembly and dispensing device assembly are properly engaged in the engaged position.

8. The pill dispensing device of claim 1, wherein the dispensing device is configured, in response to the patient pressing the dispensing button when it is unlocked, to dispense a single dose of the plurality of pills.

9. The pill dispensing device of claim 1, wherein the dispensing device is additionally provided with means for sensing whether the integrity of the dispensing capsule assembly has been compromised.

10. The pill dispensing device of claim 1, wherein the patient the user interface is configured to permit the patient to submit an electronic request via the communication means to a physician for an additional or exceptional dosage, and wherein the dispensing device is configured to unlock the dispensing button in the event of the physician's approval of such electronic request.

11. A pill dispensing system for managing and tracking the dispensing of pills to a patient, the system comprising the pill dispensing device in accordance with claim 1, and an external patient database in communication with the dispensing device, wherein the microcontroller is configured to lock or unlock the dispensing button according to the dispensing device data.

12. The pill dispensing system of claim 11, wherein the dispensing device data includes one or more of: prescription data, patient data, time from the timer, name of prescribed drug, amount of pills in a total prescription, prescription dosage (number of pills in a dose), frequency of dispensing each dose, time period between respective doses, prescription frequency, date and time of prescription activation, time/date when each dose is made available to the patient, time/date when a dose is dispensed to the patient, expiry of prescription, and time for renewing a prescription,
